(19) United States
(12) Patent Application Publication
(10) Pub. No.: US 2026/0069180 A1
(43) Pub. Date: Mar. 12, 2026

(54) SYSTEMS AND METHODS FOR ALLEVIATION OF ONE OR MORE PSYCHIATRIC DISORDERS

(71) Applicant: Tactile World Ltd, Ra'anana (IL)

(72) Inventors: Matthew WOHL, Beit Shemesh (IL); Yulia Polevaya WOHL, Beit Shemesh (IL)

(73) Assignee: Tactile World Ltd, Ra'anana (IL)

(21) Appl. No.: 19/108,461

(22) PCT Filed: Sep. 12, 2023
(86) PCT No.: PCT/IL2023/050981

§ 371 Date: Mar. 4, 2025

Related U.S. Application Data
(60) Provisional application No. 63/406,275, filed on Sep. 14, 2022.

(51) Int. Cl. A61B 5/16; A61B 5/00; A61B 5/11

(52) U.S. Cl. CPC A61B 5/165; A61B 5/1124; A61B 5/7405; A61B 5/742; A61B 5/7475

(57) ABSTRACT

Disclosed herein is a system configured to facilitate alleviation of a psychiatric disorder in a user, the system including a multimedia digital device system having at least one visual display and at least one audio output and at least one tactile output, a gesture input device configured to record a user gesture and provides the gesture to the multimedia digital device system and/or tactile input/output device (in some embodiment tactile mouse). The system includes a blindfold to temporarily neutralize the sight of the user. The multimedia digital device includes at least one processor for operating said at least one visual display and at least one tactile input/output device and said at least one audio output corresponding to command inputs of the user, interpreting signals from said sensor apparatus as being gesture-generated and for emitting a predetermined command to the computer corresponding to the gesture, and providing user information relating to one or more hand motions to be applied to the hand movable input device corresponding to a command to the user.

### FIELD OF THE INVENTION

The present invention relates to treatment of Psychiatric, Psychological and Neurological disorders.

### BACKGROUND OF THE INVENTION

Psychiatric disorders, Attention Deficit Hyperactivity Disorder (“ADHD”) etc., can be associated with the overuse of multimedia digital devices, such as computers, including the internet and electronic games. This is discussed at length in “The screens culture: impact on ADHD”, Weiss et al, (URL: http://www.ncbi.nlm.nih.gov/pmc/articles/PMC3220824/) The prevailing view of the use of computers by individuals with ADHD is that such use may exacerbate that condition, such that the use of computers by such individuals is undesirable. On the other hand some studies have shown that playing video games and watching videos has had a positive impact on intelligence and resulted in positive cognitive benefits. This is discussed at length in “The impact of digital media on children's intelligence while controlling for genetic differences in cognition and socioeconomic background”, Sauce et al, (URL: https://www.nature.com/articles/s41598-022-11341-2). The controversy surrounding the videogames and videos impact on the individuals with such disorders caused explore the influence of the tactile and haptic information channels to human brain on such disorders. There is information that engagement of tactile channel instead of visual helps train human brain to explore, structure and process information in a sequential and organized manner. By forming mental tactile images and comparing them to their existing image library, learners develop and use complex cognitive processes such as exploration, comparison, hypothetical thinking, and logical reasoning-the basis for structured and efficient learning processes (URL: https://www.icelp.info/home-3/about-us/tactile-ie/)

### SUMMARY OF THE INVENTION

The following embodiments and aspects thereof are described and illustrated in conjunction with systems, tools and methods which are meant to be exemplary and illustrative, not limiting in scope.

There is provided, in accordance with an embodiment, a system configured to facilitate the alleviation of a psychiatric disorder in a user, the system including a multimedia digital device having one or more visual displays and one or more audio outputs, a gesture input device configured to record one or more user gestures and provide one or more gestures to the multimedia digital device. The gesture input device includes a hand movable input device configured to facilitate a hand motion of the user, a sensor for recording predetermined motions of the hand movable input device and for transmitting signals corresponding to the sensed motions to the multimedia digital device, one or more hand-engageable tactile input/output devices, and a blindfold for temporarily neutralizing the sight of the user. The multimedia digital device includes one or more processors configured for operating the one or more visual displays and the one or more audio outputs corresponding to command inputs of the user, interpreting signals from the sensor apparatus as being gesture-generated and for emitting a predetermined command to the multimedia digital device corresponding to the gesture, and providing user information relating to one or more hand motions to be applied to the hand movable input device corresponding to a command to the user.

In some embodiments, the system further includes one or more hand-engageable tactile input/output devices. The one or more processors are further configure to execute input and output device software for providing tactile input and output to the user corresponding to the command input thereby.

In some embodiments, the hand movable input/output device includes an ergonomic housing formed for engagement by the hand of a user.

In some embodiments, the multimedia digital device is configured to provide a non-visual output having information including instructions for the movement of the input/output device in a sequence of one or more hand motions required to input a selected command, and an indication as to the successful completion of the sequence of hand motions required to input a selected command.

In some embodiments, the multimedia digital device is configured to provide non-visual real-time feedback to the user to facilitate the successful performance of a sequence of hand motions by the user required to input a selected command.

In some embodiments, the one or more tactile input/output devices are configured to provide a tactile output having information which includes instructions for the movement of the input device through a sequence of one or more hand motions required to input a selected command, an indication as to the successful completion of the sequence of hand motions required to input a selected command, and tactile information that represents in a tactile manner visual information that appears on the visual display.

In some embodiments, the sensor is configured to record one or more predetermined sequences of motion of the housing. Each the sequence includes two or more motions performed consecutively.

In some embodiments, the sensor is configured to sense one or more predetermined motions of the hand movable input device with respect to a biaxial system of mutually orthogonal linear axes and each motion is performed with respect to a single axis of the pair of axes.

In some embodiments, the multimedia device system is configured to approximate each motion as being along a straight line.

In some embodiments, the hand movable input/output device is a tactile computer mouse.

In some embodiments, the blindfold also includes a selectively operable signal transmitter for transmitting a signal to the multimedia digital device system so as to indicate the readiness of a user to start using the multimedia digital device.

In some embodiments, the blindfold includes one or more sensors for determining correct positioning of the blindfold on the user, and a signal transmitter for transmitting an indication to the multimedia digital device relating to the correct positioning of the blindfold over the eyes of the user.

In addition to the exemplary aspects and embodiments described above, further aspects and embodiments will become apparent by reference to the figures and by study of the following detailed description.

### BRIEF DESCRIPTION OF THE DRAWINGS

Some non-limiting exemplary embodiments or features of the disclosed user matter are illustrated in the following drawings.

FIG. 1A is a schematic illustration of a system configured for the treatment of a psychiatric disorder in a user, according to certain exemplary embodiments;

FIG. 1B is a schematic illustration of a blindfold of the system of FIG. 1A, according to certain exemplary embodiments;

FIG. 2 is a schematic illustration of an interface of the system of FIG. 1A with a user, according to certain exemplary embodiments;

FIG. 3 is a functional block diagram of a single level interface of gesture input system construction and operation, according to certain embodiments;

FIG. 4 is a functional block diagram of a multiple level interface of gesture input system construction and operation, according to certain exemplary embodiments;

FIG. 5 is a diagram of a two axis arrangement for the determination of the direction of a motion in an “UP”, “DOWN”, “LEFT”, “RIGHT” system, according to certain exemplary embodiments;

FIG. 6 is a functional block diagram of a multiple level interface of gesture input system construction and operation, according to certain exemplary embodiments;

FIGS. 7A and 7B are pictorial views of a tactile mouse, such as shown and described in any of U.S. Pat. Nos. 6,762,749 and 6,278,441, both entitled “Tactile interface system for electronic data display system,” and U.S. Pat. No. 5,912,660, entitled “Mouse-like input/output device with display screen and method for its use”, the contents of which are incorporated herein by reference;

FIGS. 7C and 7D are further schematic representations of a tactile mouse, according to certain exemplary embodiments;

FIG. 7E is a block diagram showing the main elements of a driving mechanism for the tactile display of FIG. 7A, according to certain exemplary embodiments;

FIG. 8 is a general flow diagram illustrating the basic structure of a game or exercise for training a user in the use of a gesture input device, according to certain exemplary embodiments;

FIG. 9 is a diagrammatic illustration of component motions and gestures which may be employed in the game or exercise of FIG. 8, according to certain exemplary embodiments;

FIGS. 10-13 are examples of the operation of tactile pads such as forming part of the tactile mouse illustrated in FIG. 7A, in a manner adapted to indicate to a user with covered eyes desired directions of motions, according to certain exemplary embodiments;

FIG. 14 is a diagrammatic illustration of a hybrid training exercise for a user with covered eyes of a tactile mouse incorporating a gesture input device, according to certain exemplary embodiments;

FIG. 15 is a schematic block diagram of a computer system having as separate elements a display and a gesture input device, construction and operation thereof, according to certain exemplary embodiments; and,

FIG. 16 is a schematic block diagram of a computer system having a tactile mouse in which are incorporated displays, in the form of tactile input/output devices, and a gesture input device, according to certain exemplary embodiments.

Identical, duplicate, equivalent or similar structures, elements, or parts that appear in one or more drawings are generally labeled with the same reference numeral, optionally with an additional letter or letters to distinguish between similar entities or variants of entities, and may not be repeatedly labeled and/or described. Dimensions of components and features shown in the figures are chosen for convenience or clarity of presentation and are not necessarily shown to scale or true perspective. For convenience or clarity, some elements or structures are not shown or shown only partially and/or with different perspective or from different point of views. References to previously presented elements are implied without necessarily further citing the drawing or description in which they appear.

### DETAILED DESCRIPTION

Disclosed herein is a system for the alleviation of one or more psychiatric, psychological and neurological disorders in a user, according to certain exemplary embodiments.

### Terminology

In the context of some embodiments of the present disclosure, without limiting, a gesture is a predetermined hand motion or sequence of hand motions for entering of a command to a computer device.

In the context of some embodiments of the present disclosure, without limiting, component motion is a single predetermined hand motion combining with one or more other predetermined hand motions to form a gesture.

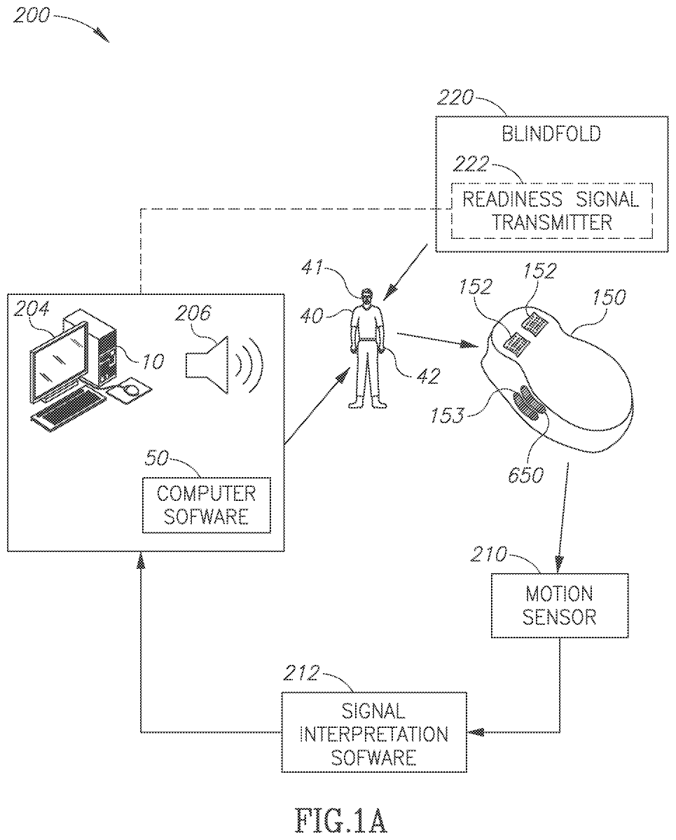

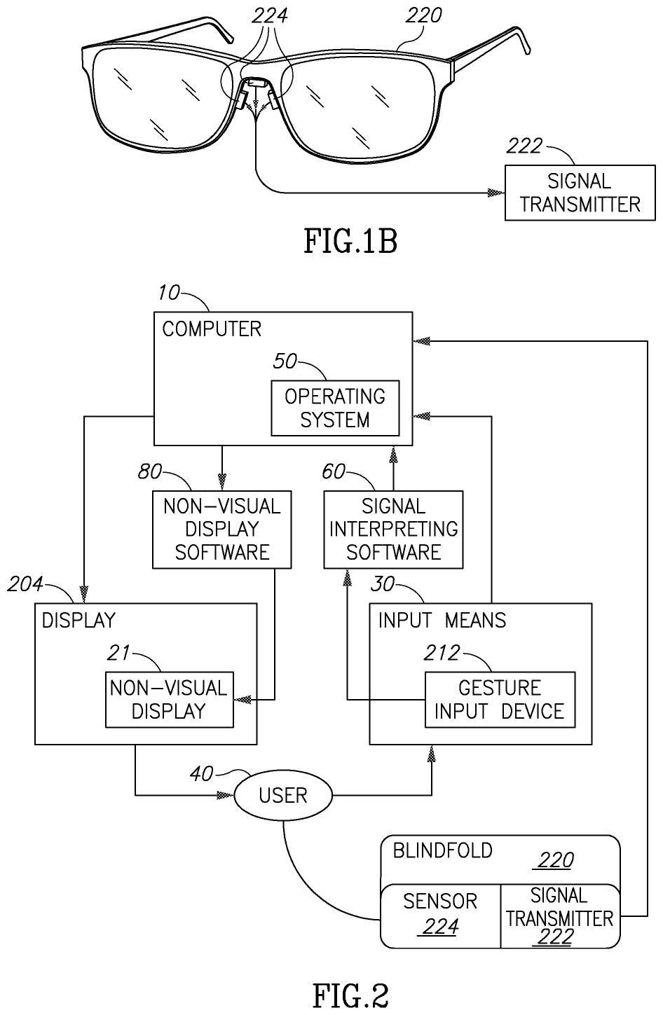

With reference to FIG. 1A, there is provided a system 200 configured for alleviation of one or more psychiatric, psychological and neurological disorders, for example, attention deficit disorder, according to certain exemplary embodiments. System 200 includes a multimedia digital device 10, such as a computer, smart phone, tablet, or the like, having associated therewith a visual display 204 and an audio output 206. For convenience, multimedia digital device 10 is often referred to as computer 10, although this is intended to include any other type of device as exemplified above.

System 200 includes a hand movable input/output device (“device”) 150 configured to record gesture generated commands performed by a user 40. In some embodiments, the system 200 includes a motion sensor apparatus 210 for recording predetermined motions of device 150 and for transmitting signals corresponding to the sensed motions to computer 10.

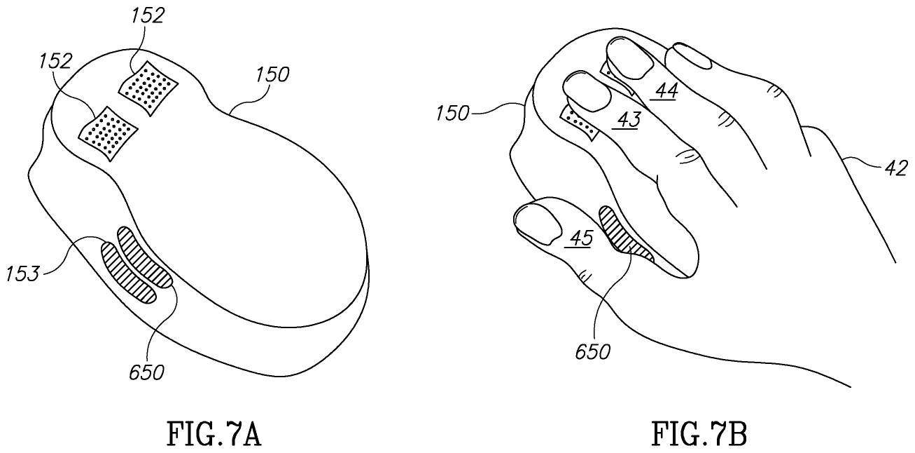

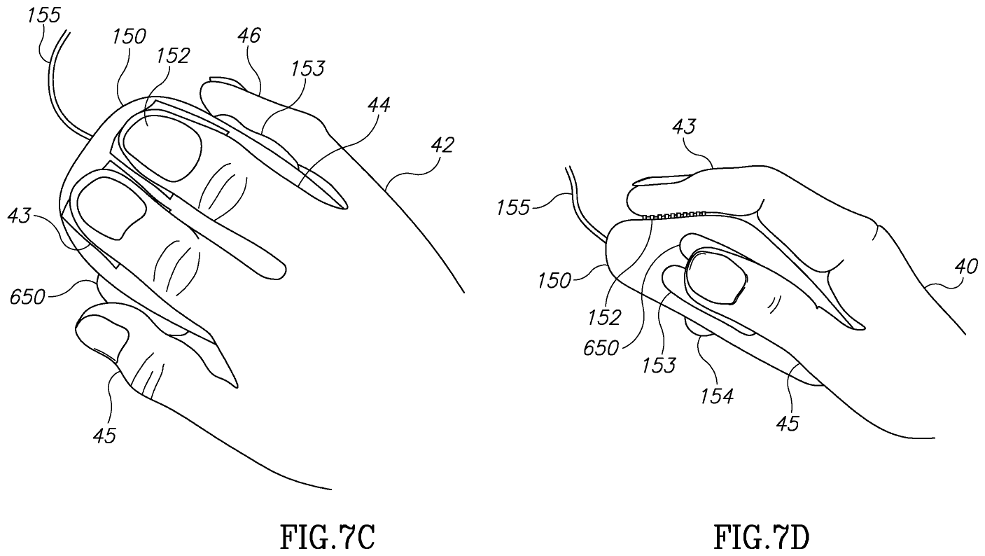

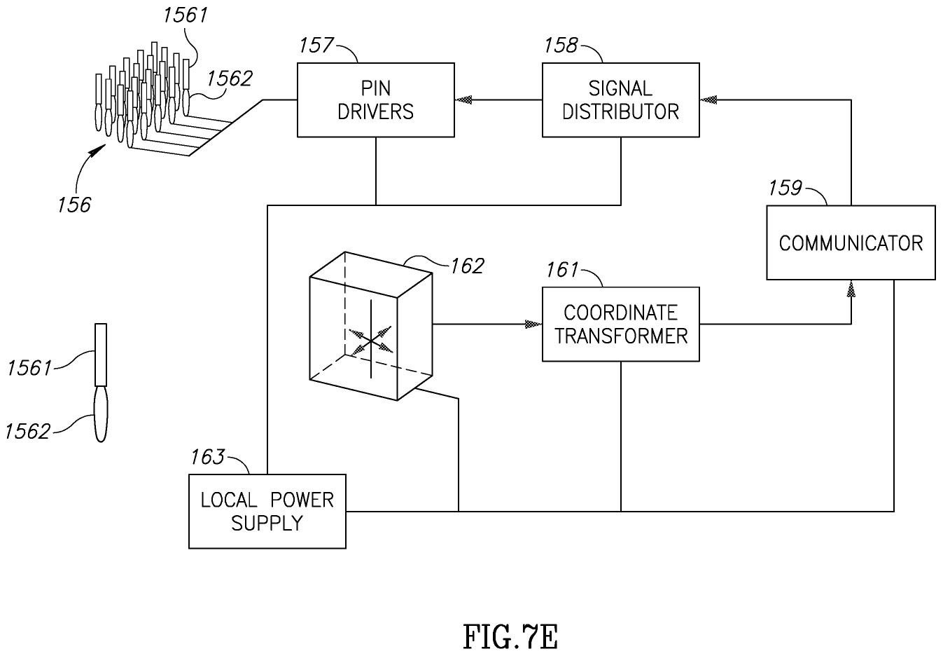

Computer 10, including one or more processors, is configured to execute a computer program product, the computer program product includes a non-transitory computer-readable storage medium having program code embodied therewith. Computer program product includes a signal interpretation software 60 for interpreting signals from motion sensor apparatus 210 as being gesture-generated and for emitting a predetermined command to computer 10 corresponding to gestures performed by a user 40. Input device 150 is ergonomically formed so as to be one hand-engageable and, as exemplified herein, has provided thereon two or more of tactile pads 152. In accordance with some exemplary embodiments, tactile mouse 150 can be manufactured in accordance with U.S. Pat. No. 5,912,660, the contents of which are incorporated herein by reference. It will be appreciated by persons skilled in the art, that tactile mouse 150, while being a single device, embodies input means and output means which together form a bi-directional tactile input/output system, the functions of which could be provided by a plurality of separate input and output devices. Tactile mouse 150 is described in greater detail hereinbelow in conjunction with FIGS. 7A-7E.

Computer 10 is configured to execute a computer program product, the computer program product includes a non-transitory computer-readable storage medium having program code embodied therewith, the program code executable by one or more hardware processors are configured to execute software for operating computer 10 so as to provide user 40 with instructions to perform one or more hand motions to be applied to hand movable input device 150. The software include instructions for executing output operations for selectively operating visual display 204 and audio output 206 corresponding to one or more hand motions performed by a hand 42 of user 40.

System 200 includes a blindfold 220 for temporarily neutralizing the sight of user 40, thereby preventing visual stimulation of user 40. In some embodiments, blindfold 220 facilitates heightening the other senses of user 40, and forces user 40 to focus on the tactile/motor interface in order to perform motor actions or gestures in order to interact with computer 10.

In some exemplary embodiments, blindfold 220 can include a signal transmitter 222 configured to transmit a signal to computer 10 so as to indicate the readiness of user 40 to start using computer 10. By way of example, transmitter 222 is configured to communicate with computer 10 via WiFi®, Near-Field Communication®, Bluetooth®, optical frequency transmitter, or the like, and can be activated by an ON-OFF sensor 224 (FIG. 1B) that detects when blindfold 220 is positioned at a desired location on a face 41 of the user 40 thereby providing an indication that the user 40 is ready to start interacting with the computer 10.

Referring to FIG. 1B, in some exemplary embodiments, blindfold 220 can include one or more sensors (“sensors”) 224 configured to detect when blindfold 220 is correctly positioned on face 41 (FIG. 1A). The detection of a correct position of blindfold 220 is operative to activate signal transmitter 222 so as to transmit a readiness indication to computer 10. In some exemplary cases, one or more sensors 224 are configured to provide an indication that blindfold 220 is correctly positioned over the eyes of user 40. For example, when children are playing interactive computer games, under adult supervision, unless the blindfold is positioned correctly such that the appropriate readiness signals are transmitted to computer 10, it will not be possible to play the game. This therefore ensures that while the supervising adult, or other professional, is able to fully follow the proficiency of the child in playing the game by use of visual display 204, user 40 must remain blindfolded at all times or the game will stop.

In some exemplary embodiments, sensors 224 can include one or more pressure sensors positioned so as to detect pressure congruent with the positioning of blindfold 220 over the eyes of user 40 generally, and more specifically, to detect the contact pressure between the blindfold 224 and the bridge of the nose of user 40. In some embodiments, sensors 224 can include one or more heat sensors for predetermined positioning on blindfold 220 so as to sense heat congruent with the heat expected to be emitted through the skin of user 40 when blindfold 220 is correctly positioned over the eyes of user 40.

With reference now to FIG. 2, which is a schematic illustration of an interface of system 200, according to certain exemplary embodiments. Computer 10 interfaces with a display 20 or other output and an input 30, such as a keyboard, computer mouse or the like, facilitated by software 50, such as operating system, executed by computer 10 to manage the interface. Software 50 can be any known operating system, additional applications, utilities and the like.

As described in conjunction with FIG. 1A, tactile mouse 150, presented here in its more general case of a handheld gesture input device, referenced 212, is seen to interact with computer 10 via signal interpretation software 60, so as to enable the input of commands to computer 10; and, as part of display 204, there is included a non-visual display 21 with associated non-visual display software 80. In some exemplary embodiments, non-visual display 21 includes one or more tactile output devices 150 as exemplified herein in FIGS. 7A-7E, and 9-13, and as described hereinbelow in detail; and audio output apparatus, denoted as 206 in FIG. 1A above. Signal interpreting software 60, non-visual display 21 with software 80 and gesture input device 31 which, some exemplary embodiments is a tactile mouse as shown and described below in conjunction with FIGS. 7A-7E, together form system 200. As described below, tactile output will be received by user 40 either as command prompts/instructions, when a command has been successfully completed, or as real time feedback when performing a gesture, or the like.

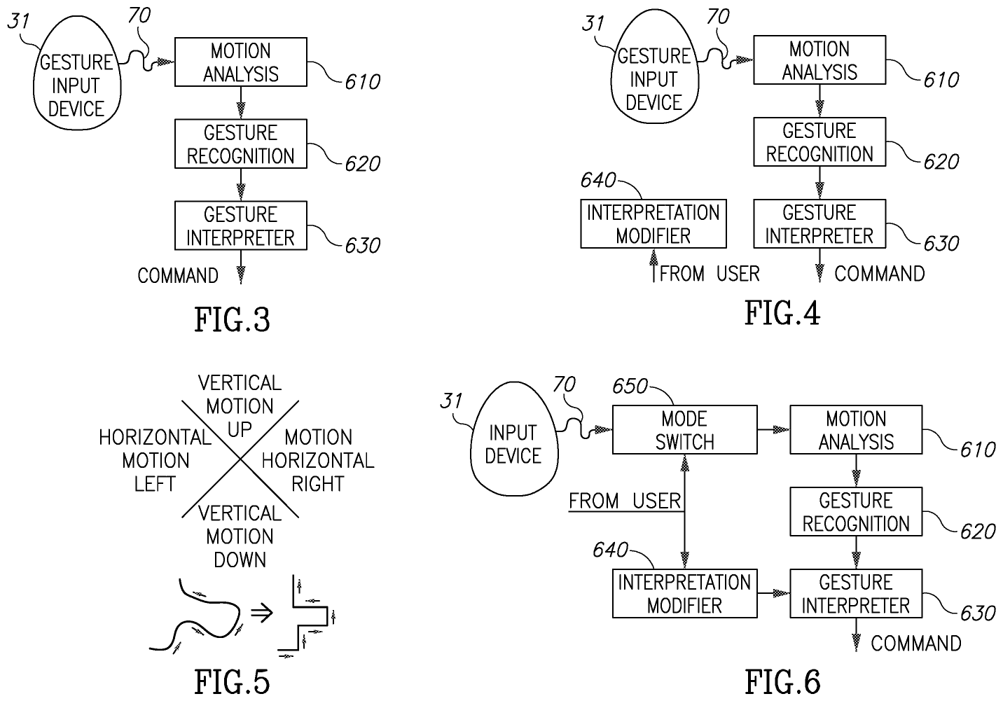

With reference now to FIGS. 3, 4 and 6, that are functional block diagrams of certain exemplary embodiments operations performed by system 200 (FIG. 1A) in response to input commands received by computer 10 (FIG. 1A) by a predetermined combination of motions or gestures.

In the illustrated functional block diagrams, there is shown gesture input device 31 of the invention, which is specifically adapted for facilitating the input of commands by gesture, as described herein. In a preferred embodiment of the invention, gesture input device 31 is tactile mouse 150 (FIG. 1A) as described herein, thereby to incorporate navigation, command and data input/selection, and tactile output, in a single, handheld device.

As illustrated, gesture input device 31 communicates with the computer 10 (FIG. 1A) via a communication channel 70 and signal interpretation software 60 (FIG. 2). Signal interpretation software 60 includes the functions of motion analysis, shown at block 610 (FIG. 3); gesture recognitions, shown at block 620; and gesture interpreter, shown at block 630, the output from which is a computer command. gesture input device 31 includes a hand-holdable housing, such as that of a computer mouse, and sensor apparatus for sensing predetermined sequences of motions of the housing with respect to a biaxial system and for transmitting signals to a computer corresponding to sensed combinations of motions of the housing. Typical sensor apparatus is exemplified by position sensors 154 in FIG. 7E, below.

As described, signal interpretation software 60 is operative to interpret the signals and to emit a predetermined command to the computer corresponding to the sensed gesture.

FIG. 3 is as functional block diagram in which each gesture, such as those described hereinbelow, corresponds to a unique command, according to certain exemplary.

Each gesture can be constituted by piecewise linear approximation of several component motions, and can be further constituted by a number of component motions, each of which must occur along one of the following two axes, as illustrated in FIG. 5. There thus result four possible motion directions, namely, left, up, right, and down. Thus, by way of example, an “L” shaped gesture includes a series of two, mutually perpendicular, component motions. More precisely there can be considered two “L” shaped gestures: the first is down and right, the second is left and up. It will be appreciated that other axial arrangements may also be considered, and that the presently described bi-axial orthogonal system is by way of example, only.

Motions of the hand-held mouse type gesture device 31 will typically not occur along a straight line in a particular direction, without deviation therefrom. Accordingly, there is provided a set of operations for the piecewise linear approximation of motions, and for interpretation thereof as being in one of the four directions in a given plane, as indicated in FIG. 5 schematically illustrates a transformation of arbitrary mouse motion to a gesture consisting of straight horizontal and vertical component motions.

In some embodiments, gestures that can be among those recognized by system 200 can include combinations or sequences of two or more sequential component motions, for example the following:
A. Left, right, up, down
B. Left+left; up+up; right+right; down+down
C. Left+right; right+left; up+down; down+up
D. Left+up; left+down; right+up; right+down
E. Up+left; up+right; down+left; down+right

In some embodiments, these twenty gestures can include a first four (Group A) of motions that are single component motion gestures, while the remaining sixteen include motions having two component motions. While it is of course possible to recognize sequences having three or four component motions, they are more complex, and may thus be difficult to remember and to perform accurately, and so are less desirable than those one and two component motion gestures listed above. However, in order to use the same gestures for the generation of different commands, and thus increase the number of available commands, the keys of a computer keyboard and/or the buttons of a tactile mouse 150 (FIG. 7A), can be used as modifiers, as described in conjunction with FIG. 4.

With reference to FIG. 4, which is a multi-level system, configured to interpret combination of motions as a two or more commands, according to certain exemplary embodiments. This is achieved by the provision of one or more gesture interpretation modifiers, referenced 640. In the present example, a single modifier only is shown, illustrated as a press button switch 153 on the tactile mouse 150 shown and described herein in FIGS. 7A-7B. Alternatively, one or more interpretation modifiers 640 may be provided by designation of keys on a conventional-type computer keyboard, for example. This allows multiplication of the twenty basic gestures exemplified above by the number of modifiers in use.

With reference to FIG. 6, which is a functional block diagram of a multiple level interface of gesture input, according to certain embodiments. In some embodiments, gesture input device 31 can be a tactile mouse 150 and includes a specific gesture mode activation switch 650 so as to prevent the system from interpreting accidental or non-specific movements of the tactile mouse 150 which were not actually intended to convey anything in particular. The switch 650 can be implemented as a button switch on the gesture input device 31 itself, as shown in FIGS. 7A-7D, or as one of the keys on a conventional keyboard.

In certain exemplary embodiments, mode selection can be affected by programming one or more of the keys of the computer keyboard.

When gesture input device 31 is implemented in tactile mouse 150 (FIGS. 7A-7E), as the tactile mouse 150 may be used both for input and output (by virtue of the tactile output devices thereof), the embodiment of gesture input device 31 in tactile mouse 150 facilitates operation of computer 10 including gesture control as described herein, without requiring user 40 to remove his hands therefrom.

In some embodiments, the system can be configured to facilitate the performance of any desired command or navigation action by predetermined gestures such as those are listed above. The following are typical commands, for illustrative purposes only.
Switch between windows
Move the cursor to the screen center, its top-left corner, others
Move the cursor to the beginning of current/previous/next line/paragraph
Read text with a speech synthesizer.
Move the cursor to a search box, favorites bar, or the like.

Referring now to FIGS. 7A-7E, there is shown, in accordance with an embodiment of the invention, gesture input device 31 (FIG. 2) as tactile mouse 150. In conjunction with FIG. 1A, tactile mouse 150, while being a single device, nonetheless embodies input means and output means which together form a bi-directional tactile input/output system, the functions of which could be provided by separate input and output devices. In some embodiments, tactile mouse 150 includes an ergonomic housing formed for engagement by a hand 42 of user 40 (FIG. 1A). In some embodiments, hand 42 interacts with tactile mouse 150 with fingers, for example thumb 45 is positioned to interact with push buttons 153 and specific gesture mode activation switch 650, fingers 43, 44 are positioned to interact with tactile displays 152, and finger 46 is positioned to interact with push buttons 153

Tactile mouse 150 is a bi-directional communication device providing a tactile output to a user via tactile displays 152, in addition to input controls via push buttons 153 used as a command entering mechanism, which may be pressed, released, clicked, double-clicked, or otherwise actuated to provide feedback to computer 10 (FIG. 1A); and a mechanism 154 (FIG. 7E) such as a roller-ball, optical sensor, or the like for sensing the position of the tactile mouse relative to its previous position.

It will be appreciated that while use of tactile mouse 150 is most convenient, embodying both data output and input in a single device, its functions may also be provided separately, for example, by provision of tactile displays 152, and input buttons/switches 153, respectively, on separate devices which cumulatively combine to provide the necessary functions input/output functions required in accordance with the present invention.

The position sensors 154 are provided to measure the variation of two or more spatial coordinates. The position of tactile mouse 150 is transmitted to the computer 10, for example via a connecting cable 155 or a wireless connection, such that each shift of the tactile mouse 150 on a work surface corresponds to a shift of the cursor of tactile mouse 150 on the visual display of the computer. These features allow the tactile mouse 150 to send input data to the computer in the same way as a conventional computer regular mouse.

As stated above, in addition to the input mechanism, a tactile mouse 150 includes one or more tactile output displays 152 for outputting data from the computer 10 to the user 40 (FIG. 1A). In some embodiments, each tactile display is a flat surface having a plurality of pins 156 which may rise or otherwise be embossed in response to output signals from the computer 10. In certain embodiments, the tactile mouse 150 has a rectangular array of mechanical pins 156 with piezoelectric actuators. In some embodiments, pins 165 can be arranged with a predetermined density of, for example, 1.5 mm distance between neighboring pins.

In certain exemplary embodiments, a driving mechanism for the tactile display 152 of the tactile mouse 150 is represented by the block diagram of FIG. 7E. The main elements of the driving mechanism are an array of pins 156, a pin driver 157, a signal distributor 158, a communicator 159, a coordinate transformer 161, a position sensing mechanism 162 and a local power supply 163 powering all electronic mechanisms of tactile mouse 150, including the tactile display 152.

As the tactile mouse 150 moves over a surface, the sensing mechanism 154 is operative to track the movements thereof. The movements of tactile mouse 150 are transformed into a set of coordinates by coordinate transformer 161 which relays the current coordinates of tactile mouse 15 to computer 10 via a communicator 159. The communicator 159 is further configured to receive an input signal from computer 10 relating to the display data extracted from the region around a tactile mouse cursor. The input signal from computer 10 is relayed to signal distributor 158 which sends driving signals to pin drivers 157. Each pin driver 157 typically drives a single pin 1561 by applying an excitation signal to an actuator 1562 such as a piezoelectric crystal, plate or the like configured to raise and lower a pin 1561.

In certain embodiments, tactile mouse 150 can be connected to the computer 10 via standard communication channels such as serial/parallel/USB connectors, Bluetooth®, wireless communication or the like. The operational interface between the tactile mouse 150 and the computer system has an input channel for carrying data from the tactile mouse 150 to the computer 10 and an output channel for carrying data from the computer to the tactile mouse 150.

Regarding the input channel, when the position sensor 154 of the tactile mouse 150 is moved along a flat working surface, the sensors measure relative displacement along two or more coordinate axes. These coordinates are converted by embedded software, into signals which are organized according to an exchange protocol and sent to the computer. Upon receiving these signals, the operating system decodes and transforms them to coordinates of the tactile mouse cursor on the computer screen. Thus, the motion of the tactile mouse cursor over the screen corresponds to the motion of the tactile mouse 150 over its working surface. The exchange protocol also includes coded signals from the tactile mouse 150 indicating actions associated with each of the input buttons such as a press signal, a release signal, a double click signal and the like.

Regarding the output channel, the output signal sent from the computer 10 to the tactile mouse 150 depends inter alia upon the coordinates of the tactile mouse cursor, and the visual contents displayed at within a predetermined range of those coordinates upon the screen. Accordingly, the tactile display 152 of the tactile mouse 150 may output a text symbol, graphical element, picture, animation, or the like. Like the regular system cursor, the tactile mouse cursor determines its own hotspot.

In some embodiments, tactile mouse 150 operated in conjunction with blindfold 220 can facilitate user 40 to make the information stored in computer 10 far more accessible to them. There are a number of reasons for this increased accessibility, notably:
The tactile mouse 150 can be effectively used for navigation among a large amount of information presented on display 20.
The movable nature of the tactile mouse 150 allows large amounts of contextual, graphical, and textual information to be displayed to the user by tactile displays 152.
Graphic objects may also be represented displayed in embossed form; for example, a black pixel can be displayed as a raised pin and a white pixel as a lowered pin. Similarly, a gray pixel can be displayed as a pin raised in an intermediate height or transformed to black or white depending on a certain threshold. Similar operations can be performed with pixels of all other colors.

### Gesture Recognition Algorithms

As described above, it is necessary to be able to distinguish between predetermined gestures and the other gesture input device motions. Such distinction can be implemented by the software in different ways. Some non-limiting illustrative examples of such implementation are as follows:

### Component Motion Recognition

It should be taken into account that mouse-like devices give relative and not absolute location and shift measurements.

### A. Continuous Motion

As per FIG. 5 different motions must be distinguished which, in certain embodiments, vary by 90°from each other:
i. x>|y| for right
ii. −x>|y| for left
iii. |x|<y for up
iv. |x|<−y for down.
   Here (x, y)—gesture input device's coordinates in an orthogonal coordinate system and |z|−absolute value of a variable z.

### Algorithm for the Implementation of Continuous Motion:

In some embodiments, (x₀, y₀) is a starting point of the device. If during N₁ and further measurements, one of the four conditions above, for example ii, is kept for current device coordinates (x, y), thus ii is the direction. N₁ is an adjustable parameter. The smaller N₁ is, the greater the user accuracy that is required. Larger values of N₁ are more convenient for people with motor skills disorder. Many other algorithms (here and below) can be used.

### B. Start Motion

This task requires differentiation between the start of a real gesture, and an accidental shift. If the number of shifts in one direction, any of i-iv above, exceed a predetermined adjustable threshold N₂, then the motion is recognized as the beginning of a gesture. Again, larger values of this parameter are recommended for users with motor disorders, but such large values may be inconvenient for use by experienced users.

### C. Stop Motion

This task requires differentiation between the termination of a real gesture, and a brief interruption in the motion, and is based on the detection of generally continuous motion. Such interruptions may be due to the user, because of errors in the mouse's motion sensor or a poor quality mouse travel surface. Accordingly, if during a specified time period N₃, no motion signals are detected from the sensor in gesture input device 31, then the gesture has stopped.

### D. Consecutive One Directional Multiple Component Gestures

The one-directional gestures, as example, are referred to mentioned above: left+left; up+up; right+right; down+down.

Each of them is a series of two (and possibly more) primitive motions separated with temporary ‘decelerations,’ which, in the context of the present invention, may be complete stops or merely slow downs. If such decelerations are allowed as separators between gestures (i.e. between two or more two-motion sequences), the speed of motion during deceleration has to be measured, thereby to determine whether the deceleration is a temporary deceleration within one gesture or a separator between two gestures.

An algorithm for use in the interpretation of consecutive one directional multiple component gestures can be based on the assumption that the motion characteristics of the gesture input device are generally uniform during a single component motion, and that a change in such characteristics cause a change in speed. Speed measurement is performed continuously during movement of the gesture input device, and a decrease in the speed by more than a predetermined adjustable parameter is considered to indicate the end of one component motion and the beginning of the next one.

### E. Consecutive Opposite Directional Gestures

Listed above as Group C is an exemplary group of opposite directional gestures, namely, left+right; right+left; up+down; down+up.

Each of these gestures is a sequence of two or more motions with stops and/or a change of motion direction such that the direction of the second motion is opposite to the first.

### F. Consecutive Mutually Perpendicular Gestures

These gestures are those mentioned above, namely, left+up; left+down; right+up; right+down and up+left; up+right; down+left; down+right.

Each of these gestures is a sequence of two or more motions with stops and/or a change of motion direction such that the direction of the second motion is perpendicular to the first.

### Algorithm for the Implementation of Mutually Perpendicular Gestures

A direction of each new vector (xₙ₊₁−xₙ, yₙ₊₁−yₙ) is compared with the known direction of the previous vector (xₙ−X₀, yₙ−y₀). If the direction of the new vector differs from the previous direction by a value approximating to 90°, a change in direction is determined to have occurred. If the new vector reaches a predetermined length when measured in terms of the number of same directional steps, this vector is determined to be a new component motion in a mutually perpendicular direction to the previous component motion.

### Training Users of the Gesture-Based System

As described hereinabove, the system of the invention is ideally suited for the visually impaired, as it relies on tactile perception for output and on manual movements for input performed while holding the gesture input device 31 of the present invention, and is preferably incorporated into a tactile mouse as shown and described hereinabove in conjunction with FIG. 7.

It is recognized, however, that the capability of entering commands into a computer by simple gestures as shown and described above, is one that because it is novel, will by definition, be initially unfamiliar to a user. Accordingly, in order to assist a new user, and particularly, although not exclusively, a visually impaired new user, in becoming familiarized with the inputting of commands as described hereinabove, by use of gestures, there are provided various training exercises so as to assist. It will be appreciated that in order to be most effective and so as to have the broadest appeal, especially to those who may not consider themselves to be computer literate, the exercises are preferably provided in the form of interactive games, thus being enjoyable, and having appeal to users of all ages.

In a further embodiment of the invention, the herein-described interactive games can employing the gesture input device 31 of the present invention may also considered to be stand alone and may be enjoyed by users without a particular learning achievement in mind.

As described above, each gesture is a sequence of motions, and apart from being interpreted as entering specific computer control or input commands, they can also be used as a manner of playing a game in which virtual spatial motions are required.

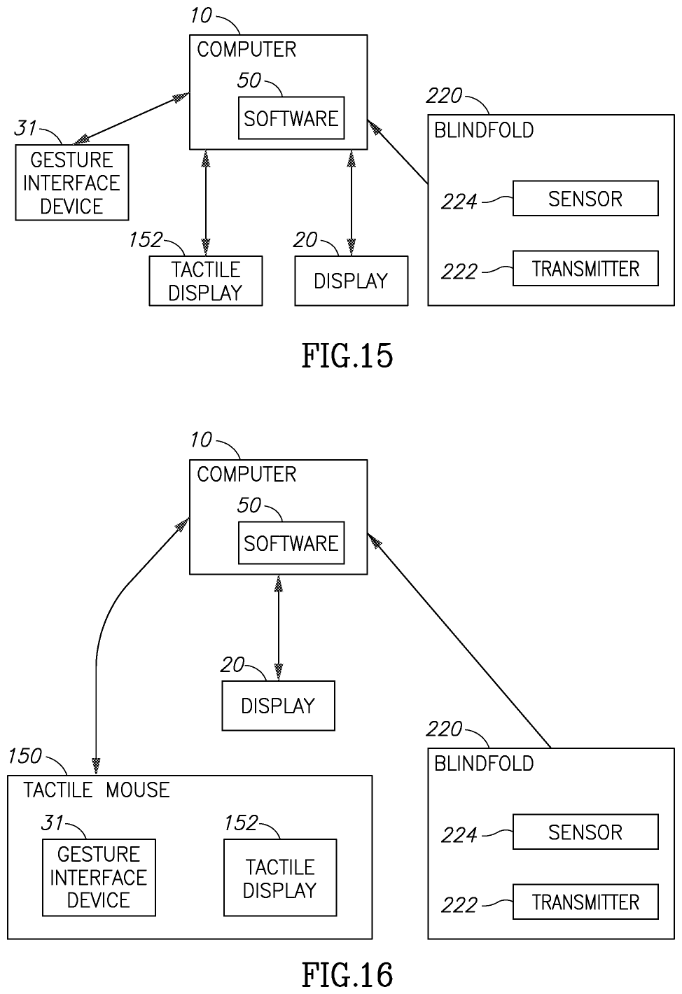

For the purpose of clarity, the training exercise or games described will be described with reference to FIGS. 15 and 16.

FIG. 15 is a schematic block diagram of a computer system, similar to that shown and described hereinabove in conjunction with FIGS. 1A and 2, and which includes computer 10 having software 50, display 20, and gesture input device 31. The display 20 may include as non-visual display means 21 (FIG. 2) one or more tactile pads 152 (FIGS. 7A-7D), integrated into a tactile mouse 150 as shown and described hereinabove in conjunction with FIGS. 7A-7D, as well as a visual display screen. It is also envisaged that both may be provided so that two or more users can either play the hereinbelow described games simultaneously, or so that one may train the other in correct use of the computer system or portions thereof.

FIG. 16 shows a similar system to that of FIG. 15, but whereas in the system of FIG. 15 the gesture input device 31 and display 20 are separate units, in the embodiment of FIG. 16, they are both incorporated into a tactile mouse 150, as shown and described hereinabove on conjunction with FIGS. 7A-7D.

The software 50 will preferably be programmed to perform the following:
(i) by use of a tactile display, to display to a user instructions for the performance of one or more predetermined gestures; these instructions may also be provided as an audio output;
(ii) to detect the performance of a gesture by the user;
(iii) to compare the gesture performed by the user with the required gesture; and
(iv) to provide feedback, preferably by means of a tactile output device but optionally also or instead, by audible means, so as to indicate to the user whether or not the gesture performed was equal to that required.

The various exercises and games described below are preferably based on the system arrangements of FIG. 15 or 16, or on variations thereof, and are merely for exemplary purposes.

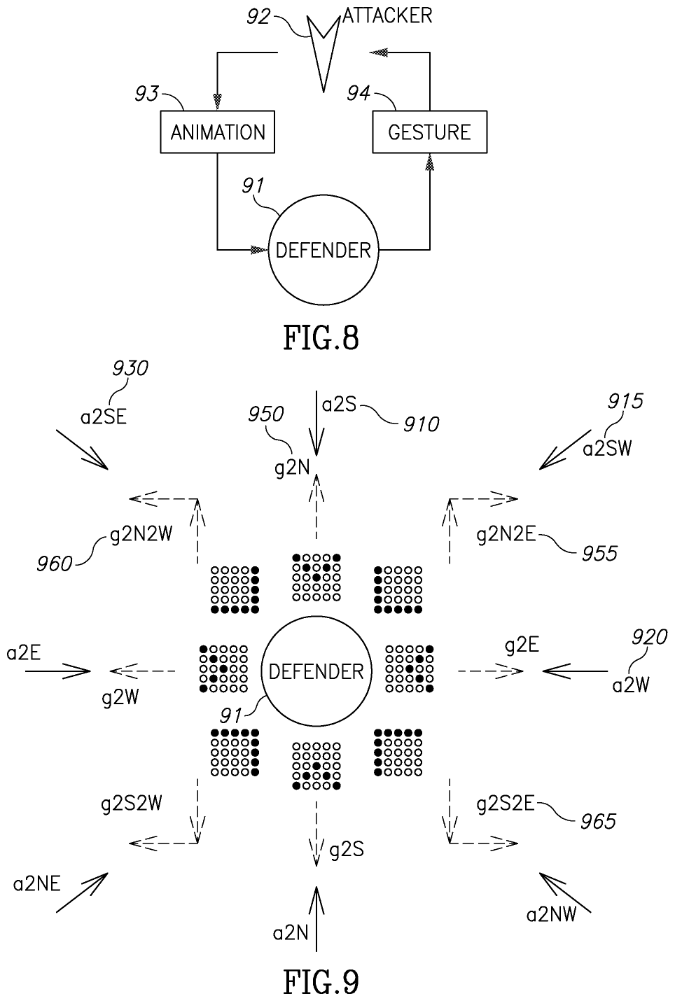

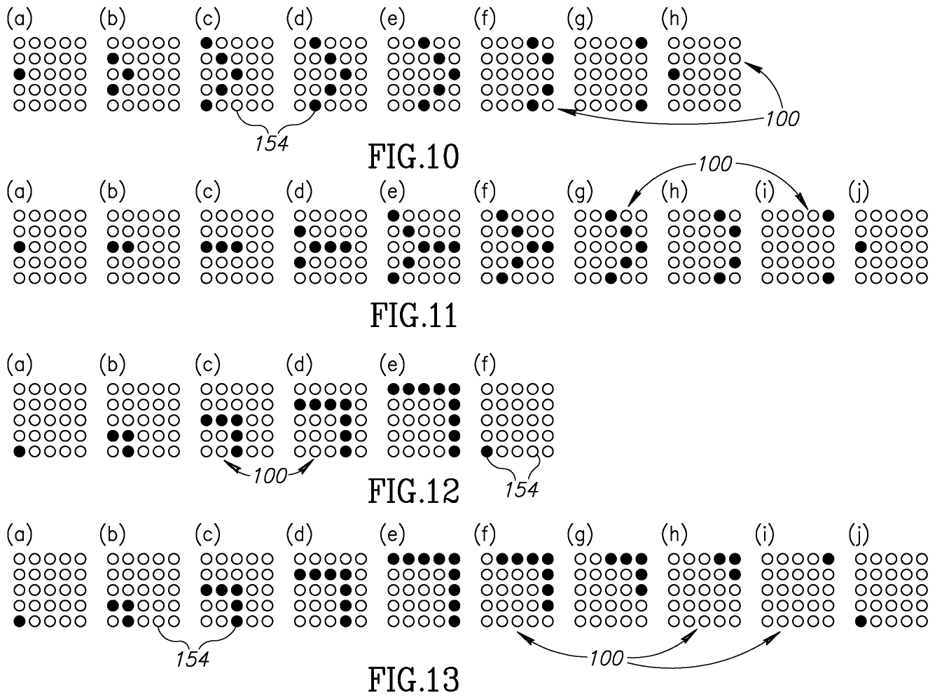

Accordingly, referring now to FIG. 8, there is illustrated a game, which may also be played by sighted users, in which a user or player is a ‘defender’ 91 who has to defend himself from an ‘attacker’ 92. Animation software 93 is employed by the system so as to activate the tactile displays 152 (FIGS. 7A-7D), for example, in a manner such as shown and described in conjunction with FIGS. 10-13, in order to provide the user with information regarding the direction of an attack.

Accordingly, with reference now to FIGS. 8 and 9, when an attack starts (attacker 92 appears from a predetermined direction and approaches the defender 91), a corresponding animation starts to run on one or more tactile output devices (FIGS. 10-13), so as to be easily perceptible by the player or defender 91. The player has to recognize an attack direction and react with an appropriate gesture 94, such as described herein. Only one gesture 94 will have the effect of beating back the attack. If the selected gesture 94 is correct, then the attack is deflected, and the player is credited with points. If the selected gesture 94 is incorrect, then the attacker will succeed in reaching the defender so as to destroy or wound it and points are subtracted. Thereafter a new attack starts either from the same or a different direction depending on the game rules. Attack directions can be selected randomly. More than one tactile output device can be used for showing animations 93. Preferably, sound effects are also provided.

In accordance with various embodiments of the invention, the rules may be modified such that each successive attack is faster, or the speed of the attacks may slow down or speed up in accordance with the skill of the player in beating off the attacks.

As seen in FIG. 9, the defender 91 has a 360° exposure to attack. Any number of attack directions can be implemented in the game. As shown by way of example in FIG. 9, eight attack directions are shown by the full, inward-pointing arrows. When viewed clockwise, the arrows are respectively referenced a2S (attack to South) 910, a2SW (attack to South-West) 915, a2W 920 and so on, all the way around until a2SE 930. Simplified versions of the game will include a decreased number of attack directions, such as:
all attacks from one direction only;
only frontal attacks: a2S 910, a2SW 915 and a2SE 930;
four directional attacks.

The defense directions, representing the gestures that need to be made by the defender 91 with gesture input device 31 in order to counter or beat off an attack have to correspond to number and directions of possible attacks. For version with eight possible directions of attack, a corresponding number of eight defense directions are shown by the broken-line arrows, respectively referenced g2N (gesture to North) 950, g2N2E (gesture to North and then to East) 955 and so on, all the way around until g2N2W 960. This does not limit the use of gestures in all possible diagonal directions, for example, gesture input device motion to North-West, North-East, and so on.

As seen, therefore, one of eight pairs of a solid line and animation shows the attack direction. For example, arrow a2SW 915 signifies an attack from the north-east to the south-west. To deflect such attack a gesture g2N2E 955, requiring the gesture input device 31 to be moved up and then right, is required. In this example, any other gesture will cause a loss for the defender, and a loss in points.

As stated above, while the animations showing attack and defense may be shown in visual form on a computer screen, they are preferably shown, either in addition or exclusively, on tactile output devices of the tactile mouse exemplified herein, for the training and enjoyment of visually impaired users. Each of the displays, referenced 100 in FIGS. 10-13, has an array of vertically displaceable pins 156, where the pins in a raised position are indicated in the drawings by solid black circles, while the pins having a circular outline only are non-raised.

Accordingly, referring now to FIG. 10, the succession of representations a-h shows how an arrow, indicated by a simple V-shape, propagates from the left or the west, and moves towards the right or to the east; the tip of the arrow is seen in representation a, the trailing ends are seen in representation g, and the tip of the next incoming arrow is seen in representation h.

FIG. 11 shows an animated arrow which has been modified for easy recognition.

FIG. 12 shows an arrow going from south east to north east.

FIG. 13 also shows an arrow going from south east to north east, but whereas the arrow in FIG. 12 seems to disappear suddenly (after representation e), the same arrow is shown in FIG. 13 to trail off gradually, as seen in representations f-j.

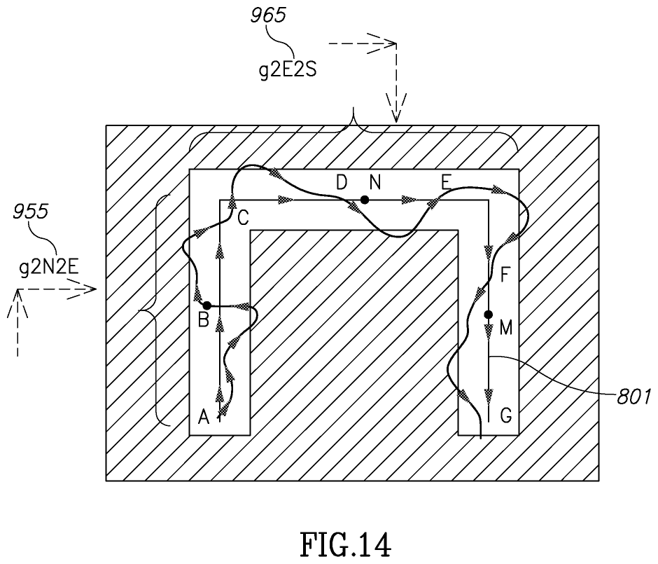

With reference now to FIG. 14, there is shown an alternative type of game, which may also serve as a tool for therapy in the treatment of certain disorders. The game involves traversing a labyrinth. It will be appreciated that the labyrinth may be formed to be as simple or as complicated as desired, and that FIG. 14 shows only a simplified portion, for illustrative purposes only. This embodiment of the invention will be described solely in conjunction with the tactile output devices of a tactile mouse as described above, serving also as gesture input device 31.

In the illustrated game, a ‘traveler’, namely user 40 (FIG. 1A), needs to traverse and exit a labyrinth. The labyrinth is shown as a white road on a black background. On the tactile output device, white is represented by the pins in a down position, while black is represented by raised pins.

Preferably, if two tactile displays are being used, one of them can show the colors (black/white) of the location of the traveler relative to the labyrinth, while another, activated as for example by animation software 93 (FIG. 8), can display possible directions for movement within the labyrinth. Clearly, if more than two tactile output devices are employed, there exist further options for the provisional of additional information to the user.

Simple movement of the tactile mouse results in a corresponding movement of the player within the labyrinth, and can enable the player to reach the goal, namely, to find his/her way out of the labyrinth. However, if the player uses correct gestures in response to animations provided at certain specific locations, by use of gestures or specific gross motions, travel can be accelerated significantly by jumping from one location to another.

In the example of FIG. 14, the player starts at location A and must reach location G. One exemplary manner to achieve this is to move as shown by line 801. This line 801 may be optionally displayed as a guide, on the tactile output device by raised pins 1561 (FIG. 7E).

If the player moves the gesture input device 31 based only on tactile perception, a possible trajectory may be as shown by the curved line A-B-C-D-E-F-G. The time that this takes may be prolonged, especially if the game rules decelerate motion when the gesture input device's cursor is out of the main road (black color).

The role of gestures in the game is to help the user anticipate and take advantage with regard to shortening in the route. For example, during motion along the vertical path from point A, the gesture g2N2E (move North and then East) 955 may be displayed to the user, signifying to the user that a bend in the route is ahead. The user may, at that time, choose to ignore the gesture, and continue gradually moving along the road, possibly following a path as shown by the curved line A-B-C-D-E-F-G. If however, he performs the indicated gesture, this will have the effect of enabling him to jump from the point where the cursor is currently located, for example B, to a point around the corner, for example N. Similarly, a gesture g2E2S may be displayed at point N, the performance of which by the user will cause him to jump around the corner, to point M.

The more quickly the player becomes used to the concept of ‘reading’ gestures and performing them correctly, the more time will be saved, leading to an ability to traverse the labyrinth more quickly. It will be appreciated that this will assist in the user becoming used to the types of motions required so as to learn how to operate a computer by using the gesture input device 31.

Additional variations to the above labyrinth game are contemplated, including but not limited to different levels of difficulty and the addition of additional, possibly more complex gestures, thereby to heighten the tactile senses of a user and further advancing therapy.

In the context of some embodiments of the present disclosure, by way of example and without limiting, terms such as ‘operating’ or ‘executing’ imply also capabilities, such as ‘operable’or ‘executable’, respectively.

Conjugated terms such as, by way of example, ‘a thing property’ implies a property of the thing, unless otherwise clearly evident from the context thereof.

The terms ‘processor’ or ‘computer’, or system thereof, are used herein as ordinary context of the art, such as a general purpose processor or a micro-processor, RISC processor, or DSP, possibly comprising additional elements such as memory or communication ports. Optionally or additionally, the terms ‘processor’ or ‘computer’ or derivatives thereof denote an apparatus that is capable of carrying out a provided or an incorporated program and/or is capable of controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry.

The processor and program may constitute the same apparatus, at least partially, such as an array of electronic gates, such as FPGA or ASIC, designed to perform a programmed sequence of operations, optionally comprising or linked with a processor or other circuitry.

The term computer, computerized apparatus or a computerized system or a similar term denotes an apparatus comprising one or more processors operable or operating according to one or more programs.

As used herein, without limiting, a module represents a part of a system, such as a part of a program operating or interacting with one or more other parts on the same unit or on a different unit, or an electronic component or assembly for interacting with one or more other components.

As used herein, without limiting, a process represents a collection of operations for achieving a certain objective or an outcome.

As used herein, the term ‘server’ denotes a computerized apparatus providing data and/or operational service or services to one or more other apparatuses.

The term ‘configuring’ and/or ‘adapting’ for an objective, or a variation thereof, implies using at least a software and/or electronic circuit and/or auxiliary apparatus designed and/or implemented and/or operable or operative to achieve the objective.

A device storing, including and/or comprising a program and/or data constitutes an article of manufacture. Unless otherwise specified, the program and/or data are stored in or on a non-transitory medium.

In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed user matter. In this regard, each block in the flowchart or block diagrams may represent a module, segment, or portion of program code, which comprises one or more executable instructions for implementing the specified logical function(s). It should also be noted that, in some alternative implementations, illustrated or described operations may occur in a different order or in combination or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘about’ and/or ‘close’ with respect to a magnitude or a numerical value implies within an inclusive range of −10% to +10% of the respective magnitude or value.

Unless otherwise specified, the terms ‘about’ and/or ‘close’ with respect to a dimension or extent, such as length, implies within an inclusive range of −10% to +10% of the respective dimension or extent.

Unless otherwise specified, the terms ‘about’ or ‘close’ imply at or in a region of, or close to a location or a part of an object relative to other parts or regions of the object.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values.

As used herein, ellipsis ( . . . ) between two entities or values denotes an inclusive range of entities or values, respectively. For example, A . . . Z implies all the letters from A to Z, inclusively.

The terminology used herein should not be understood as limiting, unless otherwise specified, and is for the purpose of describing particular embodiments only and is not intended to be limiting of the disclosed user matter. While certain embodiments of the disclosed user matter have been illustrated and described, it will be clear that the disclosure is not limited to the embodiments described herein. Numerous modifications, changes, variations, substitutions and equivalents are not precluded.

Terms in the claims that follow should be interpreted, without limiting, as characterized or described in the specification.

1. A system configured to facilitate the alleviation of a psychiatric disorder in a user, the system including:
a multimedia digital device having at least one visual display and at least one audio output;
a gesture input device configured to record at least one user gesture and provide the at least one gesture to said multimedia digital device, wherein said gesture input device includes:
a hand movable input device configured to facilitate a hand motion of the user;
a sensor for recording predetermined motions of said hand movable input device and for transmitting signals corresponding to the sensed motions to said multimedia digital device;
at least one hand-engageable tactile input/output device; and,
a blindfold for temporarily neutralizing the sight of the user,
wherein said multimedia digital device includes at least one processor configured for:
operating said at least one visual display and said at least one audio output corresponding to command inputs of the user;
interpreting signals from said sensor apparatus as being gesture-generated and for emitting a predetermined command to said multimedia digital device corresponding to the gesture, and
providing user information relating to one or more hand motions to be applied to said hand movable input device corresponding to a command to the user.

2. The system according to claim 1, further including:
at least one hand-engageable tactile input/output device,
wherein said at least one processor is further configure to execute input and output device software for providing tactile input and output to the user corresponding to the command input thereby.

3. The system according to claim 1, wherein said hand movable input/output device includes an ergonomic housing formed for engagement by the hand of a user.

4. The system according to claim 3, wherein said multimedia digital device is configured to provide a non-visual output having information including:
instructions for the movement of said input/output device in a sequence of one or more hand motions required to input a selected command; and
an indication as to the successful completion of the sequence of hand motions required to input a selected command.

5. The system according to claim 4, wherein said multimedia digital device is configured to provide non-visual real-time feedback to the user to facilitate the successful performance of a sequence of hand motions by the user required to input a selected command.

6. The system according to claim 5, wherein said at least one tactile input/output device is configured to provide a tactile output having information which includes:
instructions for the movement of said input device through a sequence of at least one hand motion required to input a selected command; and
an indication as to the successful completion of the sequence of hand motions required to input a selected command; and
tactile information that represents in a tactile manner visual information that appears on the visual display.

7. The system according to claim 6, wherein said sensor is configured to record at least one predetermined sequence of motion of said housing wherein each said sequence includes at least two motions performed consecutively.

8. The system according to claim 6, wherein said sensor is configured to sense at least one predetermined motion of said hand movable input device with respect to a biaxial system of mutually orthogonal linear axes and each motion is performed with respect to a single axis of said pair of axes.

9. The system according to claim 8, wherein said multimedia device system is configured to approximate each motion as being along a straight line.

10. The system according to claim 1, wherein said hand movable input/output device is a tactile computer mouse.

11. The system according to claim 1, wherein said blindfold also includes a selectively operable signal transmitter for transmitting a signal to said multimedia digital device system so as to indicate the readiness of a user to start using said multimedia digital device.

12. The system according to claim 1, wherein said blindfold includes:
at least one sensor for determining correct positioning of said blindfold on the user, and a signal transmitter for transmitting an indication to the multimedia digital device relating to the correct positioning of said blindfold over the eyes of the user.
